(19) United States
(12) Patent Application Publication
(10) Pub. No.: US 2026/0219247 A1
(43) Pub. Date: Jul. 30, 2026

(54) REAL-TIME MONITORING, ANOMALIES DETECTION, AND AUTOMATIC CORRECTION OF TOTAL GAS MEASUREMENTS

(71) Applicant: Schlumberger Technology Corporation, Sugar Land, TX (US)

(72) Inventors: Elena Borisova, Clamart (FR); Jean Pierre Bailly, Clamart (FR)

(21) Appl. No.: 19/057,126

(22) Filed: Feb. 19, 2025

(30) Foreign Application Priority Data
Jan. 30, 2025 (EP) 25305135.3

(51) Int. Cl. G01N 30/88; G01N 30/02; G01N 33/28

(52) U.S. Cl. CPC G01N 30/88; G01N 33/2823; G01N 2030/025; G01N 2030/8854

(57) ABSTRACT

Aspects of the present disclosure provide a method for modifying a mud-logging operation based on a total gas baseline. The method includes determining an offset between a total gas sensor and one or more gas chromatographs, supplying an operating gas containing hydrocarbons to the total gas sensor and the one or more gas chromatographs during a mud-logging operation, supplying a carrier gas to the total gas sensor and the one or more gas chromatographers from a first carrier gas source during the mud-logging operation; calculating a theoretical total gas baseline based on the offset and a measured gas chromatograph baseline; determining a drift of theoretical total gas baseline; and switching from the first carrier gas source to a second carrier gas source.

### CROSS REFERENCE TO RELATED APPLICATIONS

This application claims priority to European Patent Application No. 25305135.3, which was filed on Jan. 30, 2025, and is incorporated herein by reference in its entirety.

### BACKGROUND

The present disclosure generally relates to the field of mud logging operations in the Oil & Gas industry. During mud logging operations, gas analyzers are used to determine the composition of gas dissolved in drilling mud. Gas analyzers can include gas chromatographs (GCs) and Total Gas sensors (TGs). GCs provide the composition of the gas at regular intervals while TGs provide a continuous total gas measurement. Due to the continuous nature of the TGs, the TGs are unable to differentiate between a baseline measurement (e.g., a signal being measured when no hydrocarbons are present in the sample gas being analyzed) and the sample measurement. Accordingly, there is a need in the art for improvements in monitoring, detecting, and correcting total gas measurements.

### SUMMARY

Aspects of the present disclosure provide a method for modifying a mud-logging operation based on a total gas baseline. The method includes determining an offset between a total gas sensor and one or more gas chromatographs, supplying an operating gas containing hydrocarbons to the total gas sensor and the one or more gas chromatographs during a mud-logging operation, supplying a carrier gas to the total gas sensor and the one or more gas chromatographers from a first carrier gas source during the mud-logging operation; calculating a theoretical total gas baseline based on the offset and a measured gas chromatograph baseline; determining a drift of theoretical total gas baseline; and switching from the first carrier gas source to a second carrier gas source.

Aspects of the present disclosure provide a method for sending an alert when a drift of a total gas baseline exceeds a threshold. The method includes determining an offset between a total gas sensor and one or more gas chromatographs, supplying an operating gas containing hydrocarbons to the total gas sensor and the one or more gas chromatographs during a mud-logging operation, calculating a theoretical total gas baseline based on the offset and a measured gas chromatograph baseline, determining a drift of theoretical total gas baseline, and sending an alert when the drift exceeds a threshold.

Aspects of the present disclosure provide a method for correcting a total gas baseline drift. The method includes determining an offset between a total gas sensor and one or more gas chromatographs, determining the offset comprising supplying a carrier gas to the total gas sensor and the one or more gas chromatographs, measuring an initial baseline of the total gas sensor and an initial baseline of the one or more gas chromatographs, and calculating the offset between the initial baseline of the total gas sensor and the initial baseline of the one or more gas chromatographs, supplying an operating gas containing hydrocarbons to the total gas sensor and the one or more gas chromatographs, supplying a carrier gas from a first carrier gas source to the total gas sensor and the one or more gas chromatographers with the operating gas, calculating a drift of a theoretical total gas baseline, determining the drift is due to contamination of the carrier gas by supplying the carrier gas to the total gas sensor and the one or more gas chromatographs without the operating gas, and switching from the first carrier gas source to a second carrier gas source.

### BRIEF DESCRIPTION OF DRAWINGS

So that the manner in which the above-recited features of the disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates an exemplary mud logging operation, according to one or more embodiments.

FIG. 2 illustrates an exemplary gas analyzer system, according to one or more embodiments.

FIG. 3A illustrates an exemplary output of a gas chromatograph, according to one or more embodiments.

FIG. 3B illustrates an exemplary output of a total gas sensor, according to one or more embodiments.

FIG. 4 illustrates a method for calculating a total gas baseline, determining a drift of the total gas baseline, and correcting for the drift of a total gas baseline, according to one or more embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

The disclosure contemplates that terms such as “couples,” “coupling,” “couple,” and “coupled” may include but are not limited to welding, interference fitting, and/or fastening such as by using bolts, threaded connections, pins, clips, and/or screws. The disclosure contemplates that terms such as “couples,” “coupling,” “couple,” and “coupled” may include but are not limited to integrally forming. The disclosure contemplates that terms such as “couples,” “coupling,” “couple,” and “coupled” may include but are not limited to direct coupling and/or indirect coupling, such as indirect coupling through components such as links.

For the sake of brevity, all similar components have been given similar reference numbers with the same last two digits and a full description of such similar components may not be repeated herein. Similarly, for the sake of brevity, all like components or layers have been given the same reference numbers, and a full description of such components may not be repeated herein.

Aspects of the present disclosure provide a method for calculating a total gas baseline measurement, determining a drift of the total gas baseline measurement, and correcting for the drift of a total gas baseline measurement.

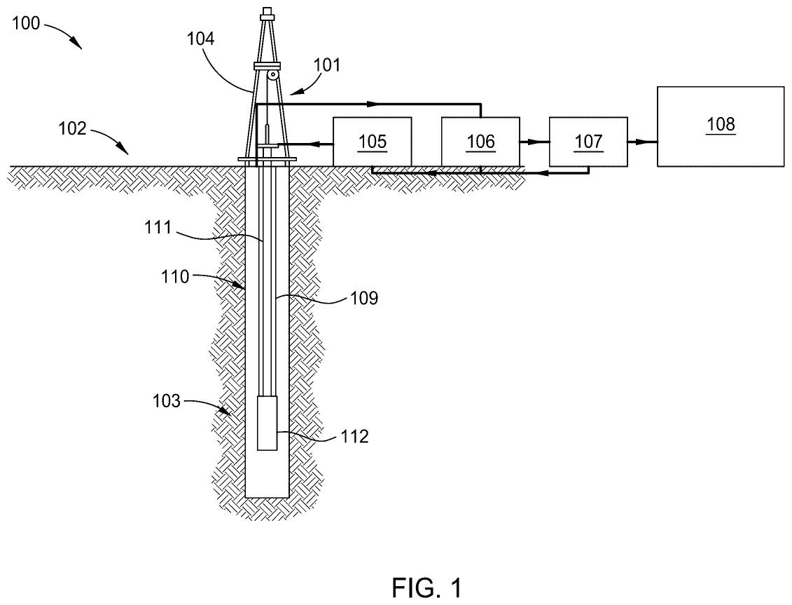

FIG. 1 illustrates an exemplary mud logging operation 100. The mud logging operation 100 includes surface equipment 101 disposed on a surface 102 above a geological formation 103.

The surface equipment 101 includes a support system 104, a drilling fluid supply 105, a drilling fluid shaker 106, a gas extractor 107, and a gas analyzer system 108.

The support system 104 supports and rotates a drill string 109 used to drill a borehole 110 in the geological formation 103. The drill string 109 includes an inner bore 111 and a drill bit 112 disposed at the end of the drill string 109. The drill bit 112 is used to drill the borehole 110.

The drilling fluid supply 105 supplies the inner bore 111 of the drill string 109 with drilling fluid. In one or more embodiments, the drilling fluid is drilling mud. In one or more embodiments, the drilling fluid is a water-based or oil-based mud. The drilling fluid flows from the drilling fluid supply 105 through the inner bore 111 of the drill string 109 to the drill bit 112. The drilling fluid exits the drill bit 112 into the borehole 110 and is allowed to flow to the surface 102. While the drilling fluid is in the borehole it may absorb some gas (e.g., “mud gas”) before it returns to the surface 102.

The drilling fluid shaker 106 is fluidly coupled to the borehole 110 such that the drilling fluid flowing to the surface 102 is directed to the drilling fluid shaker 106. The drilling fluid shaker 106 separates the drilling fluid into liquid components and solid components (e.g. cuttings).

From the drilling fluid shaker 106, the liquid components of the drilling fluid may be directed back to the drilling fluid supply 105 to be circulated back into the borehole 110 or may be flowed to to the gas extractor 107.

The gas extractor 107 separates gases that are dissolved in the drilling fluid (e.g., “mud gases”) from the drilling fluid. In one or more embodiments, the gas extractor 107 agitates the drilling fluid to separate the mud gases from the drilling fluid. In one or more embodiments, the gas extractor 107 heats the drilling fluid to separate the mud gases from the drilling fluid. In one or more embodiments, the mud gases include, but are not limited to, hydrocarbon compounds, carbon dioxide, hydrogen sulfide, helium and nitrogen. In one or more embodiments, the gas extractor 107 is fluidly coupled to the atmosphere such that the gas extractor 107 produces a gas comprising a mixture of mud gases and air. From the gas extractor 107, the drilling fluid can return to the drilling fluid supply 105 to be recirculated into the borehole 110.

From the gas extractor 107, the mud gases (and possibly the air, as discussed above) are flowed to a gas analyzer system 108. The gas analyzer system 108 is used to analyze the composition of the mud gases and is used to analyze the amount of hydrocarbons in the mud gases (e.g., “total gas content”).

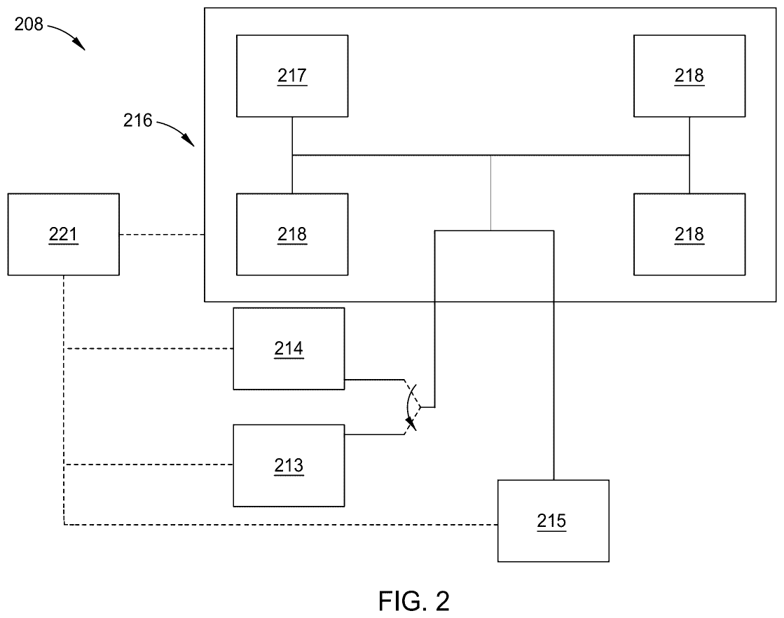

FIG. 2 illustrates an exemplary gas analyzer system 208. The exemplary gas analyzer system 208 includes a sample gas source 213, a calibration gas source 214, a carrier gas source 215, and a gas analyzer 216.

The sample gas source 213 is fluidly coupleable to the gas analyzer 216. During a mud-logging operation, the sample gas source 213 is fluidly coupled to the gas extractor 107 such that the sample gas source 213 supplies the mud gases from the gas extractor 107 to the gas analyzer 216 for analysis. In one or more embodiments, the sample gas source 213 continuously supplies mud gases to the gas analyzer 216 during mud logging operations.

The calibration gas source 214 is fluidly coupleable to the gas analyzer 216. The calibration gas source 214 supplies a calibration gas to the gas analyzer 216 so that the gas analyzer 216 can be calibrated. In one or more embodiments, the calibration gas includes gas with a known composition and volume of hydrocarbons so that the sensors can be calibrated. In one or more embodiments, the calibration gas includes minimal or no hydrocarbons.

The carrier gas source 215 supplies a carrier gas to the gas analyzer 216 so that the carrier gas serves to carry the sample through the gas analyzer 216. In one or more embodiments, the carrier gas source 215 may be used to calibrate the gas analyzer 216 (e.g., by supplying only carrier gas to the gas analyzer 216 rather than supplying carrier gas and mud gas to the gas analyzer 216). In one or more embodiments, the carrier gas source 215 is a compressor that may be installed near or far from the gas analyzer 216. In one or more embodiments, the carrier gas is air supplied by the compressor originating from an environment surrounding the compressor.

The gas analyzer 216 includes one or more total gas sensors (TGs) 217 and one or more gas chromatographs (GCs) 218. In one or more embodiments the TGs 217 are flame ionization detectors (FIDs).

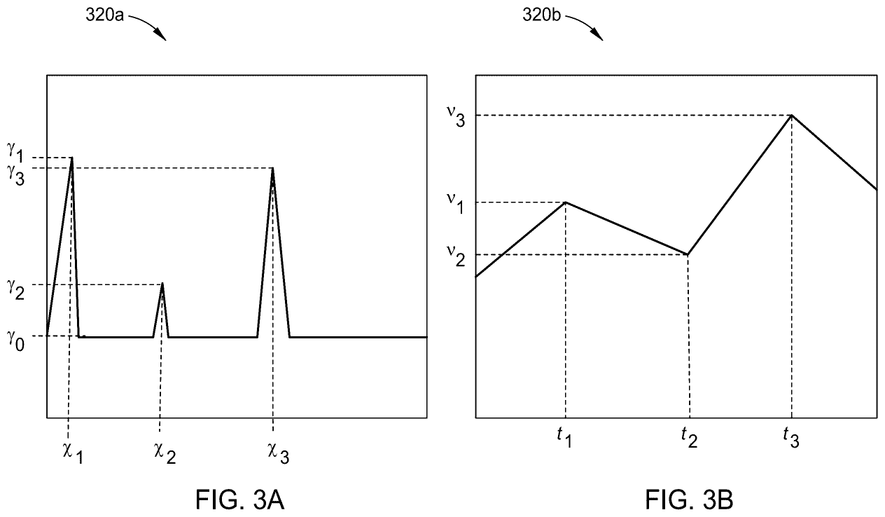

The GCs 218 receive mud gases and measure the composition of the mud gases. The GCs 218 take measurements at distinct intervals (e.g., 10 seconds(s), 20 s, 30 s, 40 s, 50 s, 60 s, or more). The GCs 218 output the components of the mud gas and the respective composition (e.g., in percentages, parts per million (ppm), etc.). An exemplary output of a GC 218 is shown in FIG. 3A. The exemplary output 320a is a chart with the y-axis being indicative of concentration and the x-axis being indicative of gas component. For example, in an exemplary output 320a of a GC 218, each peak corresponds to a measured component in the mud gas. The location of the peak on the x-axis indicates what gas the component is and the height of the peak on the y-axis indicates the concentration of said component. As an example, the illustrated mud gas includes three components located at (x₁, y₁), (x₂, y₂), and (x₃, y₃). The x-value of each indicates what the gas is and the y value determines the concentration of that gas. In between the peaks, the GC 218 measures a baseline at y₀. The baseline is the measured signal when there are no hydrocarbons are introduced to the gas analyzer 216 from the sample source 213. In one or more embodiments, the GC 218 measures a current based on the mud gases present in the sample and uses the measured current to determine the mud gas composition.

The TG 217 receives the same mud gases and measures the total hydrocarbon gas content in the sample. In one or more embodiments, the TG 217 measures the total hydrocarbons in the sample. The TG 217 takes continuous measurements. An exemplary output of a TG 217 is shown in FIG. 3B. The exemplary output 320b is a chart with the y-axis being total gas and the x-axis being time. For example, a total gas content v₁ may be determined at time t₁, a total gas content v₂ may be determined at time t₂, and a total gas content v₃ may be determined at time t₃. However, the benefit of the TG 319 is that a total gas content may be determined at any time.

In one or more embodiments, the TG 217 measures a current based on the mud gases present in the sample and uses a calibration table (e.g., a look-up table correlating current to total gas content) to determine the total gas content based on the measured current.

The TG 217 is unable to determine the difference between the components, but is rather a measurement of total gas content to track fluctuations in total gas content. Similarly, the TG 217 is not able to determine the baseline in operation. The TG 217 may measure some baseline before operation due to no sample hydrocarbons being present, but due to the nature of the TG 217 taking a continuous measurement of total gas content, the baseline is unable to be separated from the TG output during operation.

The gas analyzer system 208 may further include a control system 221. The control system 221 may include a programmable central processing unit (CPU) which is operable with a memory (e.g., non-transitory computer readable medium and/or non-volatile memory) and support circuits. The support circuits are coupled to the CPU and includes cache, clock circuits, input/output subsystems, power supplies, and the like, and combinations thereof coupled to the various components of the mud logging operation 100, to facilitate performing one or more operations of method 400. For example, in one or more embodiments the CPU is one of any form of general purpose computer processor used in an industrial setting, such as a programmable logic controller (PLC), for controlling various polishing system components and sub-processors. The memory, coupled to the CPU, is non-transitory and is one or more of readily available memory such as random access memory (RAM), read only memory (ROM), floppy disk drive, hard disk, or any other form of digital storage, local or remote.

Herein, the memory is in the form of a computer-readable storage media containing instructions (e.g., non-volatile memory), that when executed by the CPU, facilitates the generation models of subsurface regions. The instructions in the memory are in the form of a program product such as a program that implements the methods of the present disclosure (e.g., middleware application, equipment software application, etc.). The program code may conform to any one of a number of different programming languages. In one or more embodiments, the disclosure may be implemented as a program product stored on computer-readable storage media for use with a computer system. The program(s) of the program product define functions of the embodiments (including the methods and operations described herein).

Illustrative computer-readable storage media include, but are not limited to: (i) non-writable storage media (e.g., read-only memory devices within a computer such as CD-ROM disks readable by a CD-ROM drive, flash memory, ROM chips or any type of solid-state non-volatile semiconductor memory) on which information is permanently stored; and (ii) writable storage media (e.g., floppy disks within a diskette drive or hard-disk drive or any type of solid-state random-access semiconductor memory) on which alterable information is stored. Such computer-readable storage media, when carrying computer-readable instructions that direct the functions of the methods described herein, are embodiments of the present disclosure.

The various methods (such as method 400) and operations disclosed herein may generally be implemented under the control of the CPU of the control system 221 by the CPU executing computer instruction code stored in the memory as, e.g., a software routine. When the computer instruction code is executed by the CPU, the CPU conducts operations in accordance with the various methods and operations described herein. In one or more embodiments, the memory (a non-transitory computer readable medium) includes instructions stored therein that, when executed, cause the method (such as the method 400) described herein to be conducted. The operations described herein can be stored in the memory in the form of computer readable logic.

Occasionally, the carrier gas may become contaminated. For example, hydrocarbons unrelated to the mud logging operations may be introduced to the carrier gas source 215. When the carrier gas is contaminated, the effects on the GC measurement can be mitigated by taking into account the increase in the baseline. However, when the carrier gas is contaminated, the effects on the TG measurements cannot be mitigated because the baseline is not distinct and separable from the rest of the output. Thus, when the carrier gas is contaminated, the TG 217 measures an increase in total gas content and it is undeterminable whether the increase in TG measurement (e.g. drift) was from the mud gas sample or from carrier gas contamination.

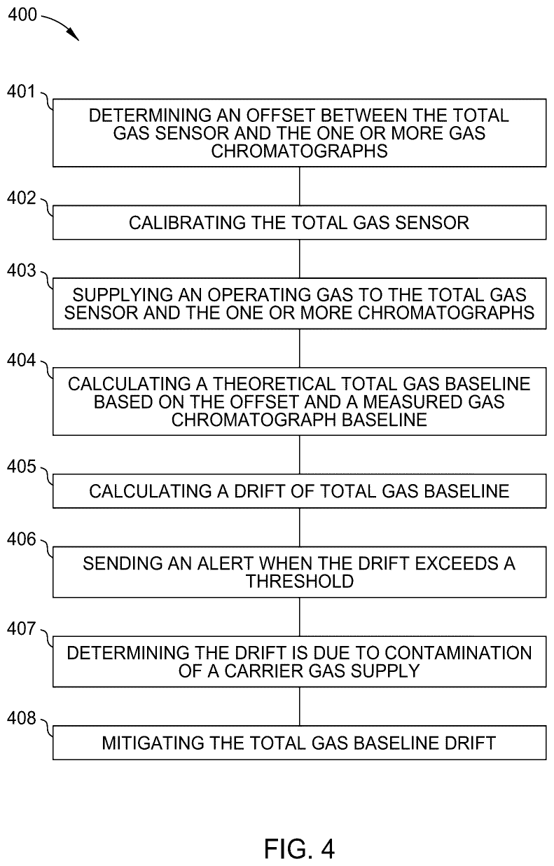

FIG. 4 illustrates a method 400 for calculating a total gas baseline, determining a drift of the total gas baseline, and correcting for the drift of a total gas baseline.

At operation 401, an offset is determined between a total gas sensor (TG) (such as TG 217 of FIG. 2) and one or more gas chromatographs (GCs) (such as GCs 218 of FIG. 2) of a gas analyzer (such as gas analyzer 218 of FIG. 2). The offset is determined by determining a difference between the baseline measurement of each of the TG and the GCs at a time zero when a sample is not supplied to the gas analyzer (e.g., determining the initial baselines). In one or more embodiments, determining the offset includes supplying a non-sample gas to the TG and one or more GCs and measuring the initial baseline of the TG and the initial baseline of the one or more GCs. In one or more embodiments, the gas used to determine the offset is a carrier gas (e.g., the carrier gas from the carrier gas source 215 of FIG. 2) to the TG and one or more GCs and measuring the baseline of the TG and the baseline of the GCs. Once the baseline of the TG and the GCs are known at a time zero, the offset will be the difference of the two. In one or more embodiments, the baseline measurements at time zero are measured in current. Accordingly, the offset would be in current, as well. As a non-limiting example, the offset between the TG and the GCs may be determined by the following formula:

$$Δ {I}_{0} = {TG}_{raw}^{0} ( {t}_{0} ) - {I}_{bsl} ( {t}_{0} )$$

In the above formula, ΔI₀ represents the offset in current,

$${TG}_{raw}^{0} ( {t}_{0} )$$

represents the current measured for the baseline by the TG at time zero and I_{bst}(t₀) represents the current measured for the baseline by the one or more GCs at time zero.

At operation 402, the TG is calibrated. In one or more embodiments, calibrating the TG includes generating a calibration table (e.g., look-up table) correlating measured current and total gas content. In one or more embodiments, the calibration table is generated using the calibration gas from the calibration gas source. As a non-limiting example, the calibration table may be formulaically represented as:

$${TG}_{conc} = F ( {TG}_{raw} ) .$$

In the above formula, TG_{conc} represents total gas content measurement, F represents the calibration table, and TG_{raw} represents the current measured by the TG.

At operation 403, a sample gas (e.g., operating gas or mud gas) is supplied to the TG and the one or more GCs. In one or more embodiments, the sample gas is supplied continuously during a mud-logging operation. The sample gas is supplied concurrently with the carrier gas. While the sample gas and carrier gas are supplied to the TG, the TG is continuously measuring a current and converting the current to total gas content. Similarly, while the operating gas and the carrier gas are supplied to the one or more GCs, the GCs are measuring a current at intervals and converting the current into gas composition measurements.

At operation 404, a theoretical total gas baseline is calculated at any operating time based on the offset and a measured GC baseline. The theoretical gas baseline may be calculated continuously based on continuous measurements from the one or more GCs. The theoretical gas baseline may be calculated based on the current measured by the one or more GCs and the offset current determined at operation 401. For example, the theoretical gas baseline in current may be the sum of the offset and the current measured by the one or more GCs. As a non-limiting example, the calculation of the theoretical gas baseline may be calculated using the following formula:

$${TG}_{raw , theo}^{0} ( t ) = {I}_{bsl} ( t ) + Δ {I}_{0}$$

In the above formula,

$${TG}_{raw , theo}^{0} ( t )$$

represents a theoretical total gas baseline at a time (t) in current, I_{bst}(t) represents the baseline measured by the one or more GCs at time (t) in current, and ΔI₀ represents the offset determined at operation 401 in current. In one or more embodiments, the theoretical total gas baseline may remain in current (as shown in the above formula) or may be converted to a total gas content measurement using the calibration table generated in operation 402.

At operation 405, a drift of theoretical total gas baseline is calculated. The drift of the theoretical total gas baseline may be calculated by determining the change in theoretical total gas baseline. In one or more embodiments, the drift may be calculated by determining the difference between theoretical total gas baselines at different moments in time. In one or more embodiments, the drift is calculated in total gas content and is thus a difference between the theoretical total gas baselines at different moments in time after the theoretical total gas baselines are converted to total gas content using the calibration table generated in operation 402. As a non-limiting example, the calculation of the drift may be calculated using the following formula:

$$( D ) ( t ) = F ( {TG}_{raw , theo}^{0} ( {t}_{x + 1} ) ) - F ( {TG}_{raw , theo}^{0} ( {t}_{x} ) )$$

In the above formula (D)(t) represents the drift as a function of time in total gas content measurement, F( ) represents the calibration table.

$${TG}_{raw , theo}^{0} ( {t}_{x} )$$

represents a theoretical total gas baseline in current at a time (tₓ), and

$${TG}_{raw , theo}^{0} ( {t}_{x + 1} )$$

represents a theoretical total gas baseline in current at a time (tₓ₊₁) sometime after time (tₓ). In one or more embodiments, the drift may be calculated in current or may be measured in total gas content using the calibration table generated in operation 402 (as shown in the above formula). In one or more embodiments, a negative drift may be indicative that contamination of the air supply was present during operation 401. In one or more embodiments, in response to determining the drift of the total gas baseline, the control system or an operator may alert a total gas baseline is drifting, modify the mud logging operation to account for the drift, investigate the drift, or correct for the drift.

At operation 406, an alert is sent when the drift exceeds a threshold. In one or more embodiments, an alert is sent to an operator. In one or more embodiments, the alert is sent to a control system (such as control system 221 of FIG. 2) utilized in the mud logging operation. The alert may alert an operator or control system that the drift has exceeded a certain threshold. In one or more embodiments, the threshold may a threshold in total gas content measurements, such as a threshold of more than 100 ppm such as between about 100 ppm and 1000 ppm or more. In one or more embodiments, threshold may be a safety threshold. For instance, the threshold may be of a value that if the drift is due to an increase in total gas composition rather than contamination of the carrier gas supply, the mud-logging operation needs to be altered or ceased for safety reasons.

At operation 407, it is determined whether the drift is due to contamination of the carrier gas supply. In one or more embodiments, determining the drift is due to contamination of the carrier gas supply includes re-checking the zero-point calibration. In one or more embodiments, rechecking the zero-point calibration includes switching the source of the TG to the carrier gas supply without the sample gas. In such embodiments, if the total gas content measured by the TG does not decrease to zero, the drift is a result of contamination of the carrier gas supply. However, if the total gas content measured by the TG decreases to zero, the drift is a result of an anomaly with the one or more GCs or is a result of total gas content of the mud gas increasing. In such cases, the mud-logging operation may be modified to correct the drift, such as by shutting down the mud-logging operation.

At operation 408, the total gas baseline drift is mitigated.

In one or more embodiments, mitigating the total gas baseline drift includes correcting total gas content measured by the TG for drift. In one or more embodiments, the total gas content is corrected by utilizing the measured baseline measured by the one or more GCs at time (t), the baseline measured by the one or more GCs at time zero, and the current measured by the TG at time (t). As a non-limiting example, the correction may be calculated by the following formula:

$${TG}_{conc}^{corr} = F ( {TG}_{raw} ( t ) - {I}_{bsl} ( t ) + {I}_{bsl} ( {t}_{0} ) )$$

In the above formula

$${TG}_{conc}^{corr}$$

represents the correction of total gas content, F( ) represents the calibration table generated at operation 402, TG_{raw} represents the measured current of the TG at time (t), I_{bst}(t) represents the baseline measured by the one or more GCs at time (t), and I_{bst}(t₀) represents the baseline measured by the one or more GCs at time zero.

With the corrected total gas content, the TG sensor more accurately determines the total gas content dissolved in the drilling fluid. With a more accurate total gas content, a control system or operator can better determine the total gas content for operational decisions, including but not limited to safety decisions.

In one or more embodiments, mitigating the total gas baseline drift includes switching the carrier gas supply to a secondary carrier gas supply that has not been contaminated.

It is appreciated that any of the above determinations and calculations may be accomplished using current and/or using total gas content measurements (e.g. ppm).

### Example Aspects

Aspect 1: A method for modifying a mud-logging operation based on a total gas baseline includes determining an offset between a total gas sensor and one or more gas chromatographs, supplying an operating gas containing hydrocarbons to the total gas sensor and the one or more gas chromatographs during a mud-logging operation, supplying a carrier gas to the total gas sensor and the one or more gas chromatographers from a first carrier gas source during the mud-logging operation; calculating a theoretical total gas baseline based on the offset and a measured gas chromatograph baseline; determining a drift of theoretical total gas baseline; and switching from the first carrier gas source to a second carrier gas source.

Aspect 2: The method of Aspect 1, wherein calculating the theoretical total gas baseline comprises adding the offset to a measured gas chromatograph baseline.

Aspect 3: The method of Aspects 1 or Aspect 2, wherein determining the drift of the theoretical total gas baseline comprises determining a difference between a first theoretical total gas baseline and a second theoretical total gas baseline, wherein calculating the first theoretical total gas baseline includes adding the offset to a first measured gas chromatograph baseline, and wherein calculating the second theoretical total gas baseline includes adding the offset to a second measured gas chromatograph baseline.

Aspect 4: The method of any of Aspects 1-3, wherein determining the offset comprises supplying the carrier gas to the total gas sensor and the one or more gas chromatographs without the operating gas, measuring an initial baseline of the total gas sensor and an initial baseline of the one or more gas chromatographs, and calculating the offset between the initial baseline of the total gas sensor and the initial baseline of the one or more gas chromatographs.

Aspect 5: The method of Aspect 4, wherein the initial baseline of the total gas sensor, the initial baseline of the one or more gas chromatographs, the theoretical total gas baseline, and the measured gas chromatograph baseline are measured in current.

Aspect 6: The method of Aspect 5, further comprising converting the theoretical total gas baseline from a current measurement into a total gas content measurement.

Aspect 7: The method of Aspect 6, wherein the theoretical total gas baseline is converted to parts per million (ppm).

Aspect 8: The method of Aspect 6 or Aspect 7, wherein converting the theoretical total gas baseline from the current measurement into the total gas content measurement comprises calibrating the total gas sensor by determining a relationship between current and total gas content of gas using a calibration gas.

Aspect 9: A method for sending an alert when a drift of a total gas baseline exceeds a threshold including determining an offset between a total gas sensor and one or more gas chromatographs, supplying an operating gas containing hydrocarbons to the total gas sensor and the one or more gas chromatographs during a mud-logging operation, calculating a theoretical total gas baseline based on the offset and a measured gas chromatograph baseline, determining a drift of theoretical total gas baseline, and sending an alert when the drift exceeds a threshold.

Aspect 10: The method of Aspect 9, wherein determining the offset comprises supplying a carrier gas to the total gas sensor and the one or more gas chromatographs without the operating gas, measuring an initial baseline of the total gas sensor and an initial baseline of the one or more gas chromatographs, and calculating the offset between the initial baseline of the total gas sensor and the initial baseline of the one or more gas chromatographs.

Aspect 11: The method of Aspect 10, wherein calculating the drift comprises calculating a first theoretical total gas baseline based on the offset and a first measured gas chromatograph baseline, calculating a second theoretical total gas baseline based on the offset and a second measured gas chromatograph baseline, and determining a difference between the first theoretical total gas baseline and the second theoretical total gas baseline.

Aspect 12: The method of Aspect 11, wherein the initial baseline of the total gas sensor, the initial baseline of the one or more gas chromatographs, the first theoretical total gas baseline, the second theoretical gas baseline, the first measured gas chromatograph baseline, and the second measured gas chromatograph baseline are measured in current.

Aspect 13: The method of Aspect 12, wherein the drift is calculated by: calibrating the total gas sensor by determining a relationship between current and concentration of gas using a calibration gas, determining the first theoretical total gas baseline and the second theoretical total gas baseline in current, converting the first theoretical total gas baseline and the second theoretical total gas baseline into concentration measurements using the relationship between current and concentration of gas using the calibration gas, and determining the difference between the converted first theoretical total gas baseline and the converted second theoretical total gas baseline.

Aspect 14: The method of any of Aspects 9-13, wherein the carrier gas is supplied concurrently with the operating gas during the mud-logging operation, and wherein the method further comprises determining the drift is due to contamination of the carrier gas.

Aspect 15: The method of Aspect 14, wherein determining the drift is due to contamination of the carrier gas comprises switching a source of the carrier gas.

Aspect 16: A method for correcting a total gas baseline drift including determining an offset between a total gas sensor and one or more gas chromatographs, determining the offset comprising supplying a carrier gas to the total gas sensor and the one or more gas chromatographs, measuring an initial baseline of the total gas sensor and an initial baseline of the one or more gas chromatographs, and calculating the offset between the initial baseline of the total gas sensor and the initial baseline of the one or more gas chromatographs, supplying an operating gas containing hydrocarbons to the total gas sensor and the one or more gas chromatographs, supplying a carrier gas from a first carrier gas source to the total gas sensor and the one or more gas chromatographers with the operating gas, calculating a drift of a theoretical total gas baseline, determining the drift is due to contamination of the carrier gas by supplying the carrier gas to the total gas sensor and the one or more gas chromatographs without the operating gas, and switching from the first carrier gas source to a second carrier gas source.

Aspect 17: The method of Aspect 16, wherein calculating the drift comprises calculating a first theoretical total gas baseline based on the offset and a first measured gas chromatograph baseline, calculating a second theoretical total gas baseline based on the offset and a second measured gas chromatograph baseline, and determining a difference between the first theoretical total gas baseline and the second theoretical total gas baseline.

Aspect 18: The method of Aspect 17, further comprising correcting the drift of the theoretical total gas baseline.

Aspect 19: The method of Aspect 18, wherein the initial baseline of the total gas sensor, the initial baseline of the one or more gas chromatographs, the first theoretical total gas baseline, the second theoretical gas baseline, the first measured gas chromatograph baseline, and the second measured gas chromatograph baseline are measured in current.

Aspect 20: The method of any of Aspects 16-19, further comprising sending an alert when the drift exceeds a threshold.

The methods disclosed herein comprise one or more actions for achieving the methods. The method actions may be interchanged with one another without departing from the scope of the claims. In other words, unless a specific order of actions is specified, the order and/or use of specific actions may be modified without departing from the scope of the claims. Further, the various operations of methods described above may be performed by any suitable means capable of performing the corresponding functions.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

While the present disclosure has been described with respect to a number of embodiments and examples, those skilled in the art, having benefit of this disclosure, will appreciate that other embodiments can be devised which do not depart from the scope and spirit of the present disclosure.

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the general principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from that described, and various actions may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for”. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for modifying a mud-logging operation based on a total gas baseline, comprising:
determining an offset between a total gas sensor and one or more gas chromatographs;
supplying an operating gas containing hydrocarbons to the total gas sensor and the one or more gas chromatographs during a mud-logging operation;
supplying a carrier gas to the total gas sensor and the one or more gas chromatographers from a first carrier gas source during the mud-logging operation;
calculating a theoretical total gas baseline based on the offset and a measured gas chromatograph baseline;
determining a drift of theoretical total gas baseline; and
switching from the first carrier gas source to a second carrier gas source.

2. The method of claim 1, wherein calculating the theoretical total gas baseline comprises adding the offset to a measured gas chromatograph baseline.

3. The method of claim 1, wherein determining the drift of the theoretical total gas baseline comprises determining a difference between a first theoretical total gas baseline and a second theoretical total gas baseline, wherein calculating the first theoretical total gas baseline includes adding the offset to a first measured gas chromatograph baseline, and wherein calculating the second theoretical total gas baseline includes adding the offset to a second measured gas chromatograph baseline.

4. The method of claim 1, wherein determining the offset comprises supplying the carrier gas to the total gas sensor and the one or more gas chromatographs without the operating gas, measuring an initial baseline of the total gas sensor and an initial baseline of the one or more gas chromatographs, and calculating the offset between the initial baseline of the total gas sensor and the initial baseline of the one or more gas chromatographs.

5. The method of claim 4, wherein the initial baseline of the total gas sensor, the initial baseline of the one or more gas chromatographs, the theoretical total gas baseline, and the measured gas chromatograph baseline are measured in current.

6. The method of claim 5, further comprising converting the theoretical total gas baseline from a current measurement into a total gas content measurement.

7. The method of claim 6, wherein the theoretical total gas baseline is converted to parts per million (ppm).

8. The method of claim 6, wherein converting the theoretical total gas baseline from the current measurement into the total gas content measurement comprises calibrating the total gas sensor by determining a relationship between current and total gas content of gas using a calibration gas.

9. A method for sending an alert when a drift of a total gas baseline exceeds a threshold, comprising:
determining an offset between a total gas sensor and one or more gas chromatographs;
supplying an operating gas containing hydrocarbons to the total gas sensor and the one or more gas chromatographs during a mud-logging operation;
calculating a theoretical total gas baseline based on the offset and a measured gas chromatograph baseline;
determining a drift of theoretical total gas baseline; and
sending an alert when the drift exceeds a threshold.

10. The method of claim 9, wherein determining the offset comprises supplying a carrier gas to the total gas sensor and the one or more gas chromatographs without the operating gas, measuring an initial baseline of the total gas sensor and an initial baseline of the one or more gas chromatographs, and calculating the offset between the initial baseline of the total gas sensor and the initial baseline of the one or more gas chromatographs.

11. The method of claim 10, wherein calculating the drift comprises calculating a first theoretical total gas baseline based on the offset and a first measured gas chromatograph baseline, calculating a second theoretical total gas baseline based on the offset and a second measured gas chromatograph baseline, and determining a difference between the first theoretical total gas baseline and the second theoretical total gas baseline.

12. The method of claim 11, wherein the initial baseline of the total gas sensor, the initial baseline of the one or more gas chromatographs, the first theoretical total gas baseline, the second theoretical gas baseline, the first measured gas chromatograph baseline, and the second measured gas chromatograph baseline are measured in current.

13. The method of claim 12, wherein the drift is calculated by:
calibrating the total gas sensor by determining a relationship between current and concentration of gas using a calibration gas;
determining the first theoretical total gas baseline and the second theoretical total gas baseline in current;
converting the first theoretical total gas baseline and the second theoretical total gas baseline into concentration measurements using the relationship between current and concentration of gas using the calibration gas; and
determining the difference between the converted first theoretical total gas baseline and the converted second theoretical total gas baseline.

14. The method of claim 10, wherein the carrier gas is supplied concurrently with the operating gas during the mud-logging operation, and wherein the method further comprises determining the drift is due to contamination of the carrier gas.

15. The method of claim 14, wherein determining the drift is due to contamination of the carrier gas comprises switching a source of the carrier gas.

16. A method for correcting a total gas baseline drift, comprising:
determining an offset between a total gas sensor and one or more gas chromatographs, determining the offset comprising supplying a carrier gas to the total gas sensor and the one or more gas chromatographs, measuring an initial baseline of the total gas sensor and an initial baseline of the one or more gas chromatographs, and calculating the offset between the initial baseline of the total gas sensor and the initial baseline of the one or more gas chromatographs;
supplying an operating gas containing hydrocarbons to the total gas sensor and the one or more gas chromatographs;
supplying a carrier gas from a first carrier gas source to the total gas sensor and the one or more gas chromatographers with the operating gas;
calculating a drift of a theoretical total gas baseline;
determining the drift is due to contamination of the carrier gas by supplying the carrier gas to the total gas sensor and the one or more gas chromatographs without the operating gas; and
switching from the first carrier gas source to a second carrier gas source.

17. The method of claim 16, wherein calculating the drift comprises calculating a first theoretical total gas baseline based on the offset and a first measured gas chromatograph baseline, calculating a second theoretical total gas baseline based on the offset and a second measured gas chromatograph baseline, and determining a difference between the first theoretical total gas baseline and the second theoretical total gas baseline.

18. The method of claim 17, further comprising correcting the drift of the theoretical total gas baseline.

19. The method of claim 18, wherein the initial baseline of the total gas sensor, the initial baseline of the one or more gas chromatographs, the first theoretical total gas baseline, the second theoretical gas baseline, the first measured gas chromatograph baseline, and the second measured gas chromatograph baseline are measured in current.

20. The method of claim 16, further comprising sending an alert when the drift exceeds a threshold.
